(19) United States
(12) Patent Application Publication
(10) Pub. No.: US 2008/0221430 A1
(43) Pub. Date: Sep. 11, 2008

(54) APPARATUS FOR MAGNETIC RESONANCE IMAGING

(76) Inventors: Hisaaki OCHI, Bellevue, WA (US); Yo Taniguchi, Kokubunji (JP); Yoshihisa Soutome, Tokyo (JP); Yoshitaka Bito, Kokubunji (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/030,893

(22) Filed: Feb. 14, 2008

(30) Foreign Application Priority Data
Mar. 7, 2007 (JP) 2007-056541

(51) Int. Cl. A61B 5/055

(52) U.S. Cl. 600/411

(57) ABSTRACT

A magnetic resonance imaging (MRI) apparatus for high-speed and high-accuracy detection of cell positions labeled with magnetic nanoparticles. A transmitter coil is controlled to generate amplitude-modulated burst RF pulses as excitation RF pulses whose amplitude is modulated by a function that repeatedly inverts the polarity of multiple high-frequency magnetic field sub-pulses separated time-wise and changes the amplitude at each polarity inversion, moreover the time interval of the amplitude-modulated burst RF pulse is set to effectively 1/(2×a first frequency), and the transmitter coil controlled so the carrier frequency of the amplitude-modulated burst RF pulse is set to a second frequency shifted substantially from the first frequency of the magnetic resonance frequency of the proton at the magnetic field strength in the MRI apparatus. The first frequency is here determined based on magnetic nanoparticle information loaded from the magnetic nanoparticle information storage unit and the magnetic resonance frequency of the proton in the static magnetic field. The MRI apparatus can in this way detect the position of cells labeled with magnetic nanoparticles, with high-speed and high accuracy.

### CLAIM OF PRIORITY

The present application claims priority from Japanese application No. JP 2007-056541 filed on Mar. 7, 2007, the content of which is hereby incorporated by reference into this application.

### FIELD OF THE INVENTION

The present invention relates to a magnetic resonance imaging (MRI) apparatus and relates in particular to an MRI apparatus for supporting inspections utilizing a contrast agent.

### BACKGROUND OF THE INVENTION

In magnetic resonance imaging with an MRI apparatus, the test subject is placed within the uniform static magnetic field generated by magnets, and an electromagnetic field is irradiated onto the test subject. A nuclear spin is excited within the subject and a nuclear magnetic resonance signal as an electromagnetic wave generated by that nuclear spin is received and used to create an image of the test subject. In recent years, imaging technology that utilizes MRI along with functional contrast agents typified by very early stage cancer detection and tracking of specific cells is the focus of much attention as a type of molecular imaging MRI. These function contrast agents mainly include positive contrast agents that make use of the short-term effect of T1 (longitudinal relaxation time) typified by gadolinium chelates; and negative contrast agents that make use of the short-term effect of T2 (transverse relaxation time) typified by SPIO (Super Paramagnetic Iron-Oxide) contrast agents. If a positive contrast agent is used then the section of the test subject with that contrast agent has high intensity distortion.

Conversely, if a negative contrast agent is used, the luminance drops in that section of the subject where the contrast agent is present, and the image appears dark. Negative contrast agents have the problem that the body contains many sections such as bones and cells with air layers that appear as dark sections on the image, so that sections of the body containing a negative contrast agent may be hard to distinguish from body sections that appear dark to begin with. Iron oxide nanoparticles such as SPIO on the other hand, offer the large advantage that their particles are extremely small (less than 100 nanometers) so that load such as toxicity induced on the body is extremely small. Iron oxide nanoparticles can also be easily inserted in specific cells and so are extremely effective for tasks such as tracking of stem cells. Due to these advantages, research efforts are being made in using positive contrast agents for imaging cells labeled with iron oxide nanoparticles typified by SPIO. This type of positive contrast imaging technology is disclosed in detail in C. H. Cunningham, T. Arai, P. C. Yang, M. V. McConnell, J. M. Pauly, and S. M. Conolly: “Positive Contrast Magnetic Resonance Imaging of Cells Labeled with Magnetic Nanoparticles”, Magnetic Resonance in Medicine, vol. 53, pp. 999-1005 (2005) and C. H. Cunningham, S. M. Conolly, I. Y. Chen, Y. Suzuki, P. C. Yang, M. V. McConnell, S. S. Gambhir, J. M. Pauly; “Off-resonance Spin Echoes for Probing the Cellular Microenvironment”, Proc. Intl. Soc. Mag. Reson. Med. 14, p 2486 (2006).

Amplitude-modulated burst pulses serving as excitation RF pulses to select a space or frequency are also known in the related art. Imaging technology utilizing amplitude-modulated burst pulses are disclosed in JP-A No. 08(1996)-308809 and JP-A No. 11(1999)-221199.

JP-A No. 08(1996)-308809 discloses excitation of a frequency or space in a comb shape by amplitude modulating a burst waveform with the sinc function; as well as shifting the comb-shaped excitation section by changing the carrier frequency of the burst waveform. This patent document also discloses a method for high-speed 2D imaging or 3D imaging by utilizing an oscillating readout magnetic field gradient and an amplitude modulated burst pulse. JP-A No. 11(1999)-221199 discloses a method for high-speed spectroscopic imaging by utilizing an oscillating readout magnetic field gradient and an amplitude modulated burst pulse.

### SUMMARY OF THE INVENTION

When cells labeled with magnetic nanoparticles are placed in a strong static magnetic field, those cells function as small magnets. FIG. 16 shows the state of the magnetic lines of force induced on the outer side of the cells labeled with magnetic nanoparticles. The direction 160 of the static magnetic field B0 is defined as the z direction. On the outer side of the cell labeled with magnetic nanoparticles, the magnetic field strength intensifies in the vicinity in the same direction of the static magnetic field, and the magnetic field strength weakens in the vicinity in the circumferential direction. FIG. 17 shows a contour map of the magnetic resonance frequency of the proton near the outer side of the cell labeled with magnetic nanoparticles. The magnetic resonance frequency becomes higher in the vicinity in the same direction as the static magnetic field, and the magnetic resonance frequency becomes lower in the vicinity in the circumferential direction. The frequency deviation is approximately plus/minus 20 ppm in the range of about 1 centimeter from the center of the cell labeled with magnetic nanoparticles. This deviation is approximately plus/minus 2.5 kilohertz at an MRI (magnetic resonance frequency of proton: approximately 128 MHz) magnetic field strength of 3 T. Unless otherwise stated hereafter, the static magnetic field strength of the MRI in these specifications is 3 T.

In typical MRI imaging, the proton in the test subject are excited and imaged in synchronization with the excitation RF frequency (On-resonance imaging). In this case, as shown in FIG. 20A, there is a region 201 in the vicinity of one centimeter within the cell labeled with magnetic nanoparticles where the signal drops and appears dark. Another approach is to set the excitation RF frequency for exciting the nucleus of hydrogen atoms within the test subject to plus 2.5 kHz from the magnetic resonance frequency of the protons in the fluid, or in other words, perform imaging by setting a frequency that is shifted away from the magnetic resonance frequency of protons in the fluid (Off-resonance imaging). In this case, as shown in FIG. 18, the excitation RF frequency on the outer side of the cell labeled with magnetic nanoparticles matches the magnetic resonance frequency of protons in fluid, along the same axis as the static magnetic field so the intensity of the image increases there, but in all other sections becomes dark because the frequencies do not match. FIG. 20 shows that image state.

The excitation RF frequency is next set to minus 2.5 kHz from the magnetic resonance frequency of protons in fluid, the protons with the test subject excited, and imaging performed (Off-resonance imaging). As shown in FIG. 19, the transmitter RF frequency matches the magnetic resonance frequency of protons in fluid, near the periphery, along the outer side of the cell labeled with magnetic nanoparticles, so that the intensity of the image increases, but in all other sections the frequencies do not match so those sections are dark. An image in this state is shown in FIG. 20C. In C. H. Cunningham, T. Arai, P. C. Yang, M. V. McConnell, J. M. Pauly, and S. M. Conolly: “Positive Contrast Magnetic Resonance Imaging of Cells Labeled with Magnetic Nanoparticles”, Magnetic Resonance in Medicine, vol. 53, pp. 999-1005 (2005), a method is disclosed for detecting the positions cells labeled with magnetic nanoparticles from two sequentially captured images which are an Off-resonance image whose excitation RF frequency is shifted in the minus direction, and an Off-resonance image whose excitation RF frequency is shifted in the plus direction. Identifying the position of the cell labeled with magnetic nanoparticles, here requires an Off-resonance image whose excitation RF frequency is shifted in the minus direction, and an Off-resonance image whose excitation RF frequency is shifted in the plus direction.

During measurement, non-uniform static magnetic fields are generated within the actual test subject, even in sections bordering on air, and the magnetic resonance frequency of the protons also deviates in those sections. In Off-resonance imaging, signals at positions where the magnetic resonance frequency matches the excitation RF frequency are detected as a high luminance positions. Utilizing just one Off-resonance image makes discriminating positions of cells labeled with magnetic nanoparticles difficult when the positions are high luminance positions caused by many localized non-uniform magnetic fields within the test subject's body. Due to this difficulty, searching for the cell (position) requires two types of Off-resonance images, that utilize the characteristic features on the outer side of cells labeled with magnetic nanoparticles, namely a position whose magnetic field intensifies along the same axis as the static magnetic field, and a position whose magnetic field strength weakens in the vicinity in the circumferential direction.

Therefore, in positive contrast imaging, imaging is usually performed of a cross section parallel to the direction of the static magnetic field. In tunnel MRI where the static magnetic field is horizontal, a coronal cross section of the test subject is usually imaged. This method has the problem that time resolution is poor compared to normal imaging since two types of off-resonance images must be acquired. For example, since labeled cells move at comparatively high speeds, the times that the two types of Off-resonance images were acquired will be different and the positions of the cells will be different in each image. So this method also has the problems that identifying the cell position is difficult and the detection accuracy is low.

Moreover, the slice cross section is preferably 5 to 20 millimeters thick. Positive contrast imaging is possible even with a thick slice cross section but has the problem that the appearance frequency of the high luminance signal (not caused by magnetic nanoparticles) becomes high and the detection accuracy drops. In the imaging sequence disclosed in C. H. Cunningham, T. Arai, P. C. Yang, M. V. McConnell, J. M. Pauly, and S. M. Conolly: “Positive Contrast Magnetic Resonance Imaging of Cells Labeled with Magnetic Nanoparticles”, Magnetic Resonance in Medicine, vol. 53, pp. 999-1005 (2005), cross section positions that are isolated from the center of the magnetic field of the MRI apparatus magnets cannot be imaged by using a slice magnetic field gradient. The reason is described using FIG. 21. FIG. 21 shows the relation between the slice position and the carrier wave of the excitation RF pulse, when selecting a coronal cross section by applying a slice magnetic field gradient in the y direction along with the excitation RF pulse. Here, the frequency on the horizontal axis is set with the magnetic resonance frequency (128 MHz) of the proton at 3 T (Tesla) as the reference zero, and the frequency deviation from that reference zero is shown on the horizontal axis. In FIG. 21, the slice magnetic field gradient is set to 5 millitesla per meter. The excitation frequency band is set to 2 kHz when the excitation RF pulse is subjected to inverse Fourier transform. When a slice magnetic field gradient of 5 millitesla per meter is applied, the magnetic resonance frequency of protons at positions separated +5 centimeters in the Y direction from the magnetic field center is +10.5 kHz from the reference zero. The excitation frequency band of the excitation RF pulse is 2 kHz so that the protons are excited within a range of 9.5 kHz to 11.5 kHz when an excitation RF pulse is applied along with the slice magnetic field gradient, and consequently a cross section is excited that is approximately 1 centimeter thick and +5 centimeters away from the center of the magnetic field.

Consider the case where the 1 centimeter thick cross section slice contains the magnetic nanoparticles. The excitation RF frequency is set to plus 2.5 kHz from the magnetic resonance frequency of protons in fluid or in other words, the case where protons in the test subject are set to a carrier frequency of 13 kHz and excited and imaged (off-resonance) is considered. At this time, the excitation RF frequency and the magnetic resonance frequency of protons in fluid match along the same axis as the standard magnetic field on the outer side of the cell labeled with magnetic nanoparticles within a cross section approximately 1 centimeter thick and separated +5 centimeters in the Y direction from the center of the magnetic field. However, when excited and imaged while set to a carrier frequency of 13 kHz, the approximately one centimeter thick cross section separated +6.2 centimeters in the Y direction from the center of the magnetic field is also excited. A signal from a typical proton with a 1 centimeter thick cross section and separated +6.2 centimeters in the Y direction penetrates into the signal acquired at this time, and makes detection of the special hydrogen atom in the vicinity of the cell labeled with magnetic nanoparticles impossible.

The same effect occurs when the excitation RF frequency is set to minus 2.5 kHz from the magnetic resonance frequency of protons in fluid and excitation and imaging of the protons in the test subject performed. Namely, the signal from the protons within cross sections other than where needed mixes into the desired signal. This unwanted signal is several times larger than the signal generated by protons near the cell labeled with magnetic nanoparticles so the desired signal becomes buried in the mixed signal and detecting it becomes impossible. Therefore imaging a cross section at positions separated from the magnetic field center of the MRI apparatus magnet is impossible when using a slice magnetic field gradient as shown in the imaging sequence disclosed in C. H. Cunningham, T. Arai, P. C. Yang, M. V. McConnell, J. M. Pauly, and S. M. Conolly: “Positive Contrast Magnetic Resonance Imaging of Cells Labeled with Magnetic Nanoparticles”, Magnetic Resonance in Medicine, vol. 53, pp. 999-1005 (2005). Unless the slice magnetic field gradient is utilized, the problem occurs that high luminosity signals not caused by the magnetic nanoparticles start appearing too frequently, and the detection accuracy deteriorates.

In C. H. Cunningham, S. M. Conolly, I. Y. Chen, Y. Suzuki, P. C. Yang, M. V. McConnell, S. S. Gambhir, J. M. Pauly; “Off-resonance Spin Echoes for Probing the Cellular Microenvironment”, Proc. Intl. Soc. Mag. Reson. Med. 14, p 2486 (2006), a method is disclosed that improves on the method in C. H. Cunningham, T. Arai, P. C. Yang, M. V. McConnell, J. M. Pauly, and S. M. Conolly: “Positive Contrast Magnetic Resonance Imaging of Cells Labeled with Magnetic Nanoparticles”, Magnetic Resonance in Medicine, vol. 53, pp. 999-1005 (2005), and that uses frequency or space selection RF pulses to detect positions of cells labeled with magnetic nanoparticles. The method in C. H. Cunningham, S. M. Conolly, I. Y. Chen, Y. Suzuki, P. C. Yang, M. V. McConnell, S. S. Gambhir, J. M. Pauly; “Off-resonance Spin Echoes for Probing the Cellular Microenvironment”, Proc. Intl. Soc. Mag. Reson. Med. 14, p 2486 (2006), also detects the cell position from two images, by using an image of frequency or space selection RF pulses shifted from the excitation RF frequency in the minus direction, and an image of frequency or space selection RF pulses shifted from the RF excitation frequency in the plus direction. C. H. Cunningham, S. M. Conolly, I. Y. Chen, Y. Suzuki, P. C. Yang, M. V. McConnell, S. S. Gambhir, J. M. Pauly; “Off-resonance Spin Echoes for Probing the Cellular Microenvironment”, Proc. Intl. Soc. Mag. Reson. Med. 14, p 2486 (2006) also discloses a method for performing spectroscopic or 3D imaging by utilizing an oscillating readout magnetic field gradient. The method in C. H. Cunningham, S. M. Conolly, I. Y. Chen, Y. Suzuki, P. C. Yang, M. V. McConnell, S. S. Gambhir, J. M. Pauly; “Off-resonance Spin Echoes for Probing the Cellular Microenvironment”, Proc. Intl. Soc. Mag. Reson. Med. 14, p 2486 (2006) performs 3D imaging or in other words, also supplies position information along the y axis and in this way conquers the problem in C. H. Cunningham, T. Arai, P. C. Yang, M. V. McConnell, J. M. Pauly, and S. M. Conolly: “Positive Contrast Magnetic Resonance Imaging of Cells Labeled with Magnetic Nanoparticles”, Magnetic Resonance in Medicine, vol. 53, pp. 999-1005 (2005) where Off-resonance imaging cannot be performed on cross sections separated from the magnetic field center. The imaging sequence disclosed in C. H. Cunningham, S. M. Conolly, I. Y. Chen, Y. Suzuki, P. C. Yang, M. V. McConnell, S. S. Gambhir, J. M. Pauly; “Off-resonance Spin Echoes for Probing the Cellular Microenvironment”, Proc. Intl. Soc. Mag. Reson. Med. 14, p 2486 (2006), uses frequency or space selection RF pulses to perform off-resonance imaging within a thick cross section, and applied phase encoding along the y axis so can obtain information within thin cross sections. However, this method just like C. H. Cunningham, T. Arai, P. C. Yang, M. V. McConnell, J. M. Pauly, and S. M. Conolly: “Positive Contrast Magnetic Resonance Imaging of Cells Labeled with Magnetic Nanoparticles”, Magnetic Resonance in Medicine, vol. 53, pp. 999-1005 (2005), cannot perform off-resonance imaging on a single cross section separated from the magnetic field center. C. H. Cunningham, S. M. Conolly, I. Y. Chen, Y. Suzuki, P. C. Yang, M. V. McConnell, S. S. Gambhir, J. M. Pauly; “Off-resonance Spin Echoes for Probing the Cellular Microenvironment”, Proc. Intl. Soc. Mag. Reson. Med. 14, p 2486 (2006) always requires 3D imaging in order to acquire off-resonance information on cross sections separated from the magnetic field center.

So when the desired cross section (cross section containing magnetic nanoparticles) is at a position separated from the magnetic field center, this method has the problem that information from unwanted areas is acquired during the 3D imaging so that there are delays in the imaging time. This method of C. H. Cunningham, S. M. Conolly, I. Y. Chen, Y. Suzuki, P. C. Yang, M. V. McConnell, S. S. Gambhir, J. M. Pauly; “Off-resonance Spin Echoes for Probing the Cellular Microenvironment”, Proc. Intl. Soc. Mag. Reson. Med. 14, p 2486 (2006) therefore also has the problem that time resolution is poor compared to the usual imaging of the related art. Since the labeled cells move at comparatively high speeds, the times that the two types of off-resonance images are acquired will be different and the positions of the cells will be different in each image so that this method also has the problems that identifying the cell position is difficult and the detection accuracy is low.

In view of the above problems with the related art, this invention has the object of providing an MRI apparatus capable of high-speed and high-accuracy detection of cell positions labeled with magnetic nanoparticles.

One aspect of the apparatus of this invention includes: a magnet for generating a static magnetic field; a transmitter coil for generating excitation RF pulses to apply to the subject placed in the static magnetic field; a magnetic field gradient generator unit for a gradient magnetic field overlapping onto the magnetic field; a receiver coil for detecting nuclear magnetic resonance signals emitted from the subject; a magnetic particle information storage unit for storing information about the magnetic nanoparticles; a control unit for generating amplitude-modulated RF pulses by forming multiple high-frequency magnetic field sub-pulses separated time-wise as excitation burst RF pulses whose polarity is repeatedly inverted and whose amplitude is modulated at each polarity inversion by a mathematical function, and for controlling the transmitter coil by setting the time interval of the amplitude-modulated burst RF pulses to 1/(2×a first frequency), and so that the carrier frequency of the amplitude-modulated burst RF pulse is effectively set to a second frequency shifted from the first frequency, as the resonant frequency of the protons at the magnetic field strength in the static magnetic field; and an information processor unit for forming image data based on the nuclear magnetic resonance signal detected by the receiver coil and, characterized in that the control unit sets the first frequency based on information about magnetic nanoparticles loaded from the magnetic particle information storage unit, and the magnetic resonance frequency of the proton at the magnetic field strength of the static magnetic field.

This invention is capable of high-speed, high-accuracy detection of positions of cells labeled with magnetic nanoparticles from one type of Off-resonance image. This method allows identifying a cell position even when the labeled cell is moving at a comparatively high speed so that an MRI apparatus capable of very early stage cancer detection and tracking specific cells can be achieved utilizing a safe functional contrast agent.

### BRIEF DESCRIPTION OF THE DRAWINGS

FIG. 1 is a concept view of the MRI apparatus;

FIG. 2 is a block diagram showing the overall MRI apparatus of this invention;

FIG. 3 is a diagram showing the pulse sequence of this invention;

FIG. 4 is a drawing showing the amplitude-modulated burst RF pulse of this invention;

FIG. 5 is sine waves showing the profile along the frequency axis and also along the time axis of the amplitude-modulated burst RF pulse of this invention;

FIG. 6A is sine waves showing the profile along the frequency axis of the amplitude-modulated burst RF pulse of this invention;

FIG. 6B is sine waves showing the profile along the frequency axis of the amplitude-modulated burst RF pulse of this invention;

FIG. 7A is a drawing showing the reconstructed image of this invention;

FIG. 7B is a drawing showing the reconstructed image of this invention;

FIG. 7C is a drawing showing the reconstructed image of this invention;

FIG. 7D is a drawing showing the reconstructed image of this invention;

FIG. 8 is a flow chart for showing the image reconstruction process flow in this invention;

FIG. 9 is waveforms showing pulse sequence variations of this invention;

FIG. 10 is waveforms showing pulse sequence variations of this invention;

FIG. 11 is waveforms showing pulse sequence variations of this invention;

FIG. 12 is a flow chart showing the frequency setting flow in this invention;

FIG. 13A is waveform and a profile along the frequency axis;

FIG. 13B is a waveform variation of the amplitude-modulated burst RF pulse of this invention;

FIG. 13C is waveform variations of the amplitude-modulated burst RF pulse of this invention;

FIG. 14 is waveforms showing pulse sequence variations of this invention;

FIG. 15A is a waveform and profile along the frequency axis and along the time axis for the amplitude-modulated burst RF pulse utilized in this invention;

FIG. 15B is a waveform and profile along the frequency axis and along the time axis for the amplitude-modulated burst RF pulse utilized in this invention;

FIG. 15C is a waveform and profile along the frequency axis and along the time axis for the amplitude-modulated burst RF pulse utilized in this invention;

FIG. 16 is a drawing showing the state of the magnetic rays excited on the outer side of the cell labeled with magnetic nanoparticles;

FIG. 17 is a contour drawing of the magnetic resonance frequency of the proton near the outer side of the cell labeled with magnetic nanoparticles;

FIG. 18 is a drawing showing an area containing two luminosities arrayed along the direction of the static magnetic field in the vicinity of the cell labeled with magnetic nanoparticles;

FIG. 19 is a drawing showing an area containing two luminosities arrayed intersecting the direction of the static magnetic field in the vicinity of the cell labeled with magnetic nanoparticles;

FIG. 20A is a drawing of the reconstructed image;

FIG. 20B is a drawing of the reconstructed image;

FIG. 20C is a drawing of the reconstructed image; and

FIG. 21 is a graph showing the relation between the carrier frequency for the excitation RF pulse and the slice position.

### DETAILED DESCRIPTION OF THE PREFERRED EMBODIMENTS

The preferred embodiments of the MRI apparatus of this invention are described next in detail. This invention is not limited by these embodiments.

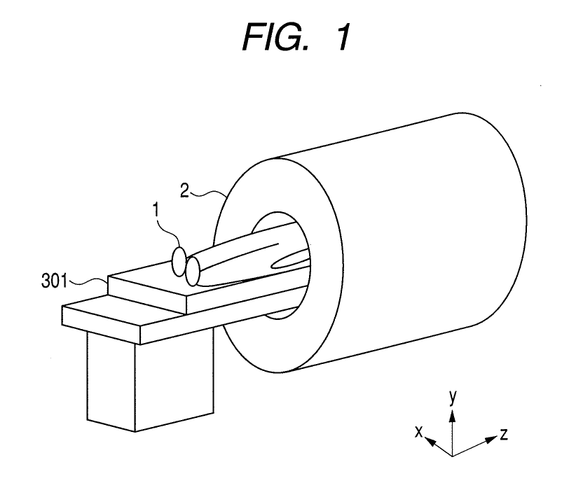

The entire structure of the MRI apparatus of this invention is herewith described. FIG. 1 is a view showing the exterior of the MRI apparatus. In this figure, the Z axis is the direction of the static magnetic field. The magnetic resonance imaging apparatus in FIG. 1 includes a horizontal field type magnet 2. In this figure, the subject 1 in a prone position on the table 301 is inserted for imaging into the imaging space within the bore of the magnet 2.

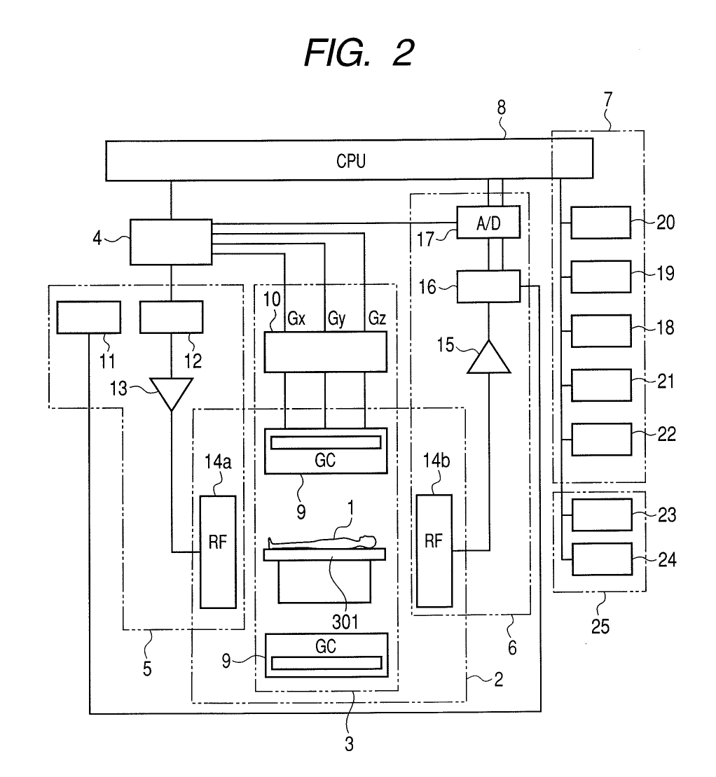

An example of the MRI apparatus of this invention is described next based on FIG. 2. FIG. 2 is a block diagram showing the entire structure of an embodiment of the MRI apparatus of this invention. This MRI apparatus acquires cross sectional image of the subject by utilizing the NMR phenomenon. Therefore as shown in FIG. 2, the MRI apparatus includes a static magnetic field generator 2, a magnetic field gradient generator 3, a transmitter system 5, a receiver system 6, a signal processor 7, a sequencer 4, and a central processing unit (CPU) 8.

If the static magnetic field generator 2 is the perpendicular magnet field type then a static magnetic field is generated in a direction intersecting the body axis in the space around the subject 1, and if the horizontal magnetic field type then a static magnetic field is generated uniformly with the body axis so that a permanent magnet, resistive electromagnet or a superconducting make up the electromagnet static magnetic field generating source positioned around the body 1.

The magnetic field gradient generator 3 includes the gradient field coils 9 wound along the three axes X, Y, Z that are the MRI apparatus coordinate system (static coordinate system), and a magnetic field gradient power source 10 for driving each of the gradient field coils. The sequencer 4 described later on, drives the magnetic field gradient power source 10 to apply the magnetic field gradients Gx, Gy, Gz in the three X, Y, Z directions. During imaging, the slice surface for the subject 1 is set by applying a slice direction magnetic field gradient pulse (Gs) in a direction perpendicular to the slice and, a phase encoding gradient pulse (Gp) and a frequency encoding gradient field pulse (Gf) are applied to the remaining two directions perpendicular to the slice direction, and their respective position information is encoded into an echo signal.

The sequencer 4 is a control means for repeatedly applying a specified pulse sequence as the magnetic field gradient pulse and an RF magnetic field pulse (hereafter called “RF pulse”). The sequencer 4 utilizes control by the CPU8, to send the various commands required for collecting cross sectional image data on the subject 1, to the transmitter system 5, magnetic field gradient generator 3, and the receiver system 6.

The transmitter system 5 irradiates RF pulses onto the subject 1 in order to induce nuclear magnetic resonance in the nuclear spin of the atoms making up the body tissues of the subject 1. The transmitter system 5 therefore includes an RF frequency generator 11 and a modulator 12 and an RF amplifier 13 and an RF coil (transmit coil) 14a on the transmitter side. The modulator 12 modulates the RF pulses output from the RF frequency generator 11 at a timing specified by commands from the sequencer 4. The RF amplifier 13 then amplifies these amplitude-modulated RF pulses and by supplying them to the RF coil 14a installed in the vicinity of the subject 1, irradiates the RF pulses onto the subject 1.

The receiver system 6 detects the echo signal (NRM signal) emitted by the nuclear magnetic resonance of the atom nuclear spin in the body tissues of the subject 1. The receiver system 6 includes an RF coil (receive coil) 14b on the receiver side, a signal amplifier 15 an orthogonal phase detector 16, and an A/D converter 17. The RF coil 14b installed near the subject 1 detects the NMR signal induced in the subject 1 by the magnetic waves irradiated from the RF coil 14a on the transmitter side. The signal amplifier 15 then amplifies the signal and the orthogonal phase detector 16, divides it into two intersecting signal types at the timing specified by commands from the sequencer 4. The A/D converter 17 then converts these signals into digital quantities, and sends them to the signal processor 7.

The signal processor 7 stores and displays the processing results and performs the various data processing tasks. This signal processor 7 includes external storage devices such as an optical disk 19 and a magnetic disk 18, as well as a display 20 such as a CRT. When the data from the receiver system 6 is input into the CPU8, that CPU8 processes the signal and reconstructs the image, and along with showing those results as a cross sectional image of the subject 1 on the display 20, records the results on the external storage device such as the magnetic disk 18. The magnetic particle information is stored in the ROM21 or the RAM22.

An operating console 25 is used to input all types of control information for the MRI apparatus and control information on processes executed by the signal processor 7. The operating console 25 is a trackball or a mouse 23 and a keyboard 24. This operating console 25 is installed near the display 20, and the operator control the various types of processing on the MRI apparatus interactively by way of the operating console 25 while viewing the display 20.

The transmit side RF coil 14a and the gradient field coil 9 are installed in the static magnetic field space in the static magnetic field generator 2, facing the subject 1 if the static magnetic field generator 2 is the perpendicular magnet field type; and are installed to enclose the subject 1 if the static magnetic field generator 2 is the horizontal magnetic type. The RF coil 14b on the receive side is installed facing the subject 1 or enclosing the subject 1.

The atomic type used for imaging in current MRI apparatus and whose clinical use is spreading is the proton which is a structural element that makes up most of the subject's body. By imaging the information relating to the spatial distribution of the proton density, and the spatial distribution of the relaxation time in the excitation state, the state or the functions of subject sections such as the human cranium, stomach or limbs can imaged two-dimensionally or three dimensionally.

### First Embodiment

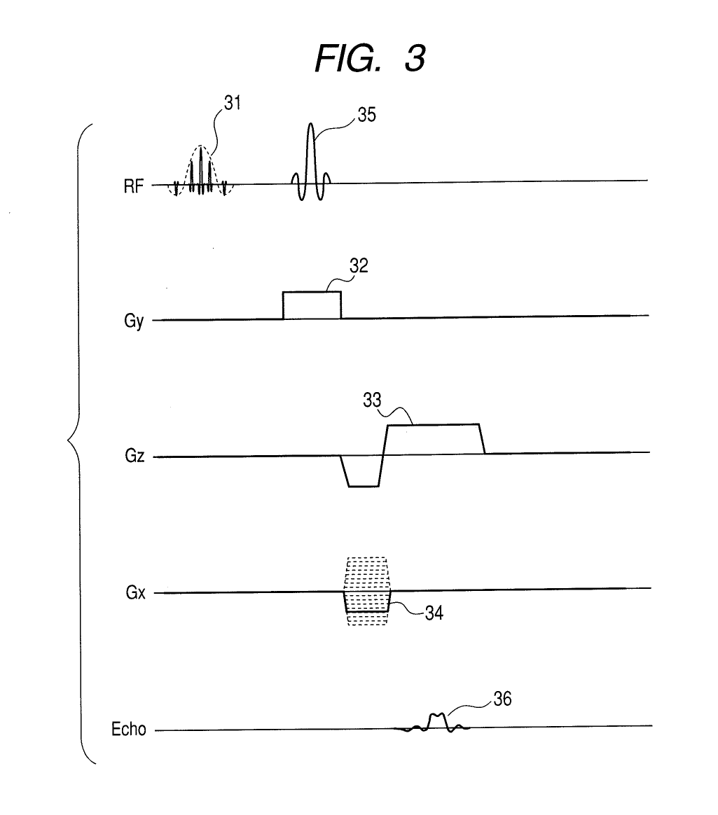

The static magnetic field strength of the MRI apparatus is 3 T (tesla). The pulse sequence of this invention is first of all described using FIG. 3. In FIG. 3, the horizontal axis is the time. An amplitude-modulated burst RF pulse 31 made up of five sub-pulses is first of all irradiated onto the subject. The carrier frequency (RF-center-frequency) of this amplitude-modulated burst RF pulse 31 is set to a frequency shifted plus 2.5 kHz from the magnetic resonance frequency (128 MHz) of the proton in the fluid. This frequency shift is a value 2/100000 (20 ppm) multiplied by the magnetic resonance frequency of the proton. This value 20 ppm is the SPIO characteristic value. This characteristic value changes when the magnetic particle is changed. Namely, the burst pulse carrier frequency is generally set to frequency shifted by (magnetic resonance frequency of proton at the magnetic field strength of the MRI apparatus)×(characteristic value of the magnetic nanoparticles).

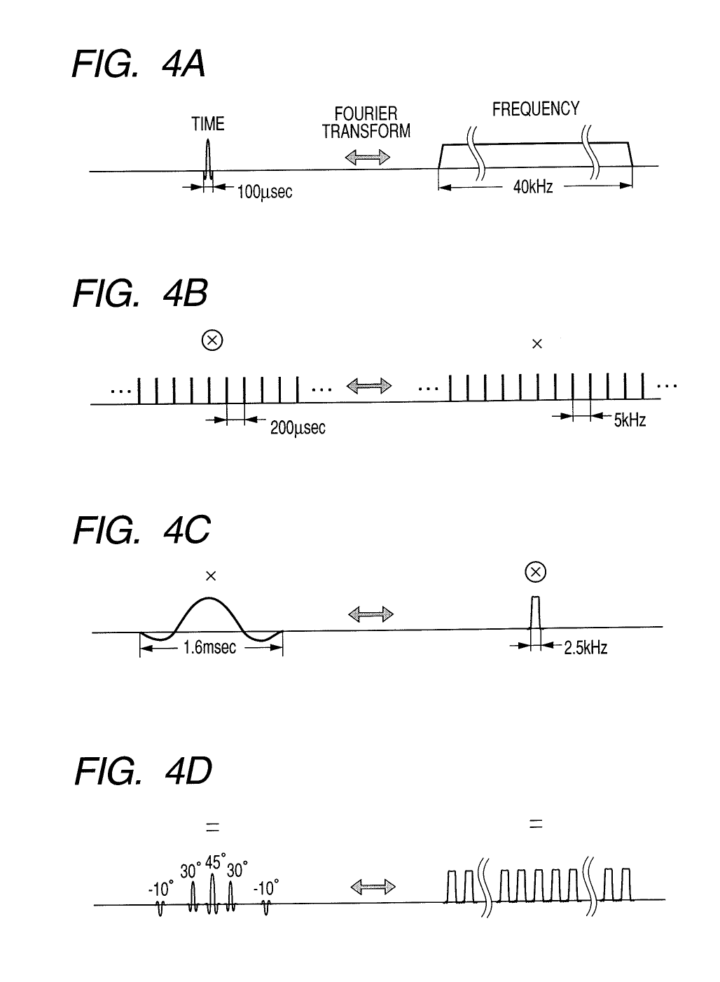

The action on the frequency axis of the amplitude-modulated burst RF pulse is described using FIG. 4 through FIG. 6. FIG. 4 shows design indicators for the amplitude-modulated burst pulse using the sinc function. The drawings on the left in FIG. 4 show the waveform in the time region, and the drawings on the right show a profile of the time region waveform in the frequency range after inverse Fourier transform. A burst pulse (d) amplitude-modulated by the sinc function is expressed as the product of the convolutions (a), (b), (c).

In FIG. 4, the time interval on the sine wave profile in the frequency range is designed to be 5 kHz, and the frequency bandwidth for one profile in the sine (comb) wave profile is designed to be 2.5 kHz. Here, the 200 microsecond time interval for the amplitude-modulated burst RF pulse is 1/(2×2.5 kHz), or in other words, is calculated as 1/(2×20 ppm of (magnetic resonance frequency of proton at static magnetic field strength of MRI apparatus). This 20 ppm is the characteristic value of the SPIO, and changes when the type of magnetic particle is changed. In other words, the time interval for the burst pulse is generally set to essentially be 1/(2×(magnetic resonance frequency of proton at static magnetic field strength of MRI apparatus)×(magnetic nanoparticle characteristic value). If the setting accuracy is within an error range of plus/minus 10 percent then there will basically be no adverse effects on the test accuracy. This magnetic nanoparticle characteristic value (magnetic particle information) is stored in the magnetic nanoparticle information storage unit in the signal processor of the MRI apparatus. This magnetic nanoparticle information is selected according to the type of magnetic nanoparticle contrast agent used in the exam, and loaded (read-out) during imaging.

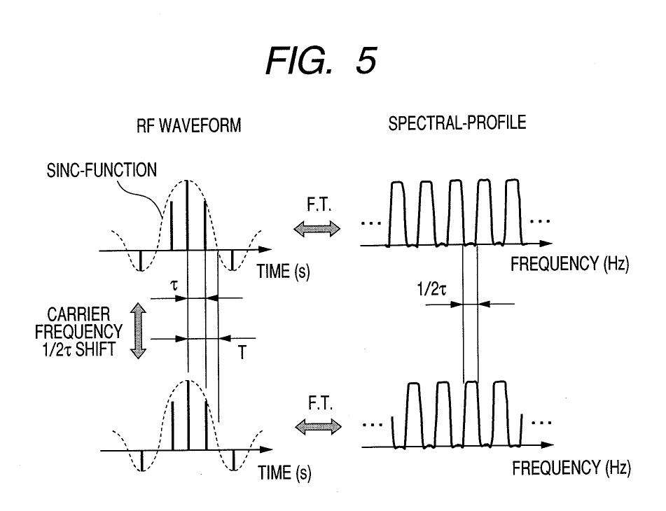

Each flip angle among the five sub pulses are respectively set to −10°, 30°, 45°, 30°, and −10°, so that the flip angle will reach approximately 90° in the excitation region when irradiating the amplitude-modulated burst RF pulse (on the subject). FIG. 5 shows the action in the frequency range, when the carrier frequency of the amplitude-modulated burst RF pulse was changed. When the carrier frequency is shifted 1/2 τ (2.5 kHz in this embodiment), the peaks and dips of the comb profile interchange precisely with each other in the frequency range. The comb excitation profile can also be shifted to the right or left in the frequency range by changing the phase of the sub pulse carrier wave under specified conditions in spite of shifting the carrier.

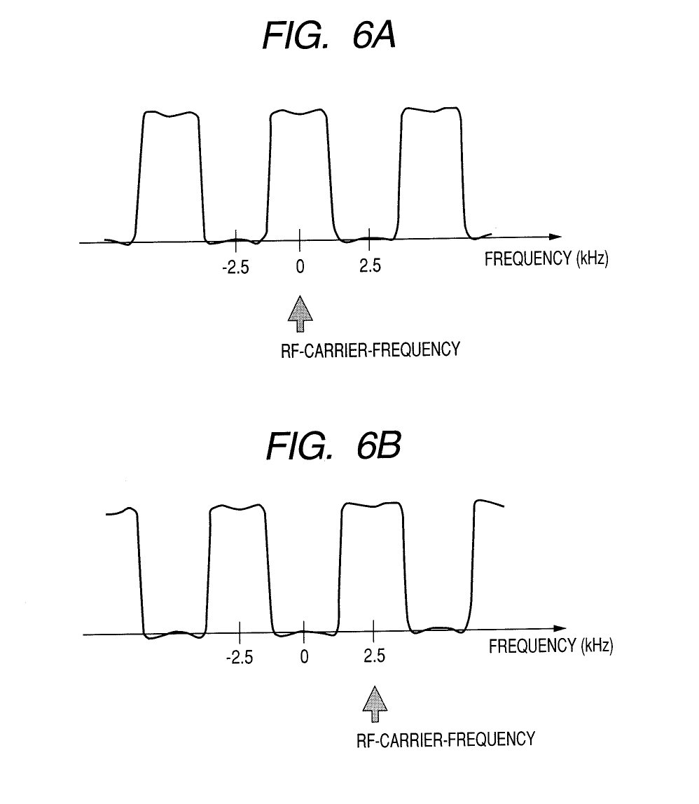

When the carrier wave phase of the five sub pulses for example are all burst pulses of 0 degrees and when the phases of the carrier wave are in order, burst pulses of 180 degrees, 180 degrees, 0 degrees, 180 degrees, and 180 degrees then the peaks and dips of the comb excitation profiles exactly interchange with each other within that frequency range. FIG. 6 is an enlarged view of FIG. 5, where the horizontal axis is the frequency. Using a magnetic resonance frequency (128 MHz) for the proton in fluid at 3 T as a reference of zero, the frequency shift from that reference zero is shown on the horizontal axis and the extent of excitation on each frequency is shown on the vertical axis. As shown in FIG. 6A, the proton in the fluid possessing a magnetic resonance frequency in a range from −1.25 kHz to 1.25 kHz, is excited when the carrier frequency is set to the magnetic resonance frequency (128 MHz) of proton in fluid at 3 T and imaged (on-resonance imaging).

There is no excitation of proton having a magnetic resonance frequency within a range of 1.25 kHz to 3.75 kHz and a range of −3.75 kHz to 1.25 kHz. In other words, those proton in regions near the outer side of the cells labeled with magnetic nanoparticles are not excited. As shown in FIG. 6B on the other hand, those protons having a magnetic resonance frequency in a range from 1.25 kHz to 3.75 kHz and a range from −3.75 to −1.25 kHz, are excited when the carrier frequency is set to the magnetic resonance frequency for proton in fluid +2.5 kHz at 3 T and imaging performed (off-resonance imaging). Protons having a magnetic resonance frequency in fluid in a range between −1.25 kHz to 1.25 kHz are not excited. In other words, protons are excited in a range near the outer side of those cells labeled with magnetic nanoparticles. Both protons in regions in a direction concentric with the static magnetic field direction, and protons in regions near the periphery can be excited one time.

The present invention in this way utilizes an amplitude-modulated burst RF pulse where the carrier frequency was shifted a specified value from the magnetic resonance frequency of the hydrogen proton at the static magnetic field strength of the MRI apparatus as the excitation RF pulse. The extent of this frequency shift is set based on the magnetic particle information loaded from the magnetic particle information storage unit, and the magnetic resonance frequency of the protons in the static magnetic field. The magnetic resonance frequency of protons at the static magnetic field strength of the MRI apparatus is 128 MHz and the SPIO magnetic particle information is 20 ppm when the contrast agent is SPIO and the MRI apparatus static magnetic field strength is 3 T, so the frequency shift is calculated as 128 MHz×20 ppm or approximately 2.5 kHz.

If this type of RF burst pulse is utilized then the magnetic resonance frequency of the protons in fluid near the outer side of cells labeled with magnetic nanoparticles will match the excitation band in the frequency range of the excitation RF pulse so that the protons in that region are excited. On the other hand, the frequency of protons in fluid in areas without a non-uniform static magnetic field which is most other sections, is the magnetic resonance frequency of protons in fluid at 3 T, which does not match the excitation band in the frequency region of the excitation RF pulses and so protons in fluid in that region are not excited.

Namely, the luminosity of protons in fluid in regions without non-uniform magnetic fields where luminosity becomes high in the conventional imaging method, can be imaged at low luminosity and the luminosity of protons in fluid in regions near the outer side of the cells labeled with magnetic nanoparticles where luminosity is low in conventional imaging methods, becomes high. As shown in FIG. 6B, the pulse interval in the burst pulse frequency range is approximately twice the amount that the carrier frequency was shifted from the magnetic resonance frequency of protons at the static magnetic field strength of the MRI apparatus. Here, the pulse interval of the burst pulse within the time region as shown in FIG. 4B, is 1/(pulse interval of the burst pulse at the frequency range) The pulse time interval of the amplitude-modulated burst RF pulse is therefore set to approximately, 1/(2×(amount that carrier frequency was shifted from the magnetic resonance frequency of protons at the static magnetic field strength of the MRI apparatus).

In amplitude-modulated burst RF pulses designed under the above conditions, the size of the excitation profile in that frequency range is nearly zero at a frequency whose reference is zero (128 MHz) on a bilaterally symmetrical waveform. If the size of the excitation profile is 1 at ±2.5 kHz, then any excitation at the reference zero frequency is less than 1/10,000 normal excitation. In positive contrast imaging it is extremely important not to cause excitation in fluid (magnetic resonance frequency of 128 MHz) not containing a non-uniform magnetic field. The total signal of fluid in areas without non-uniform static magnetic fields is 10,000 times larger than the total signal caused by protons around cells labeled with magnetic nanoparticles. The desired signal might become buried within the signal if mixed with fluid from areas without non-uniform static magnetic fields so that detecting the desired (excitation) signal becomes impossible.

In cases where SPIO is the magnetic particle, then 20 ppm (20 ppm of the magnetic resonance frequency of protons in static magnetic field of the MRI apparatus) will prove an effective amount to shift the carrier frequency. If the carrier frequency is shifted too much then the area where excited for the Off-resonance imaging becomes small, making detecting of the desired area difficult. Conversely, if the carrier wave frequency is not shifted enough then signals in the region where the excitation RF frequency matches the magnetic resonance frequency will occur due to non-uniform static magnetic fields (such as at boundaries with air, etc.) for other than the magnetic nanoparticles, resulting in poor detection efficiency. This 20 ppm value is a characteristic value in SPIO, and changes according to the type of magnetic particle. The carrier frequency of the burst pulse is in other words, usually set to a frequency shifted only by the (magnetic resonance frequency of protons at the MRI apparatus magnetic field strength)×(characteristic value of the magnetic nanoparticles). If the magnetism of the magnetic nanoparticles in the contrast agent is stronger than SPIO, then shifting the carrier frequency by more than 20 ppm will prove effective. Moreover, if the magnetism of the magnetic nanoparticles in the contrast agent is weaker than SPIO, then shifting the carrier frequency by less than 20 ppm will prove effective. The pulse interval at these times is generally set to attain 1/(2×(magnetic resonance frequency of protons at the MRI apparatus static magnetic field strength×the characteristic value of the magnetic nanoparticles).

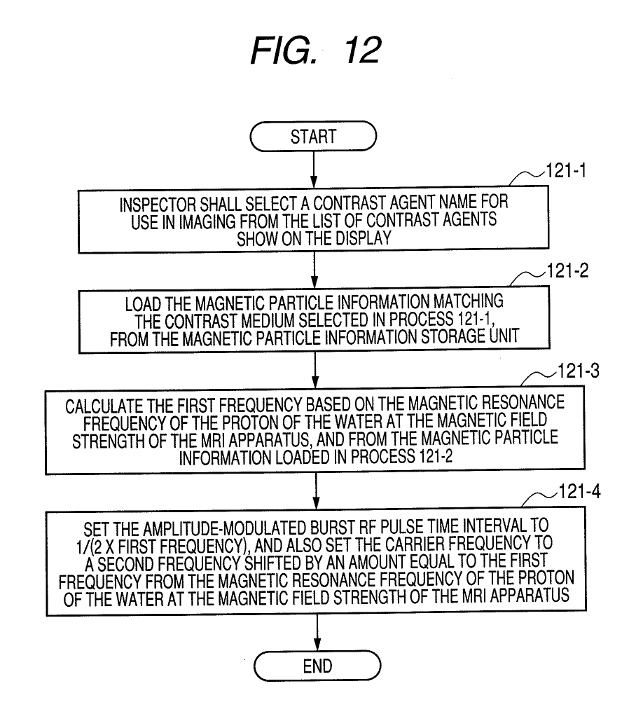

In cases where the magnetic particle is SPIO, and the static magnetic field strength of the MRI apparatus is 3 T then, and the magnetic resonance frequency of the protons at the MRI apparatus static magnetic field strength is 128 MHz, then the SPIO magnetic particle information (characteristic value of magnetic nanoparticles) is 20 ppm so that calculating at 1/(2×(128 MHz)×20 ppm)) yields approximately 200 microseconds. FIG. 12 is a drawing showing the flow when setting the carrier frequency, and the pulse time interval of the amplitude-modulated burst RF pulse, based on the magnetic particle information. The magnetic particle information (characteristic value of magnetic nanoparticles) is placed beforehand in a table format to correspond to the contrast agent names, and that table is stored in the magnetic particle storage unit.

In FIG. 12, the operator first of all, selects a name among the contrast agents used in the imaging on the display. Magnetic particle information matching the selected contrast agent names is then loaded from the magnetic particle storage unit. A first frequency is then calculated based on the magnetic particle information that was loaded (characteristic value of magnetic nanoparticles) and the magnetic resonance frequency of the hydrogen proton in fluid at the static magnetic field strength of the MRI apparatus. The pulse time interval of the amplitude-modulated burst RF pulse is then set to 1/(2×first frequency), and carrier frequency is set to a second frequency shifted by an amount equal to the first frequency from the magnetic resonance frequency of the protons in fluid.

In FIG. 3, after applying an amplitude-modulated burst RF pulse 31, a 180° pulse 35 is applied along with a slice magnetic field gradient 32, to invert the protons within the desired cross section of the subject, and disperse the phase of the protons outside the cross section. The carrier frequency of the 180 pulse 35 is set to the magnetic resonance frequency (128 MHz) of protons in fluid at 3 T.

The 180° pulse 35 to be applied, is identical to the pulse utilized in conventional imaging so that unlike the imaging sequence disclosed in C. H. Cunningham, T. Arai, P. C. Yang, M. V. McConnell, J. M. Pauly, and S. M. Conolly: “Positive Contrast Magnetic Resonance Imaging of Cells Labeled with Magnetic Nanoparticles”, Magnetic Resonance in Medicine, vol. 53, pp. 999-1005 (2005), thin cross sections separated from the magnetic field center can also be imaged. Moreover, the imaging cross section can be tilted for oblique imaging just the same as in conventional imaging. The imaging sequence disclosed in C. H. Cunningham, T. Arai, P. C. Yang, M. V. McConnell, J. M. Pauly, and S. M. Conolly: “Positive Contrast Magnetic Resonance Imaging of Cells Labeled with Magnetic Nanoparticles”, Magnetic Resonance in Medicine, vol. 53, pp. 999-1005 (2005) can also image thin cross sections that are separated away from the magnetic field center by utilizing the 180° pulse in conventional imaging. However, if attempting off-resonance imaging with the 90° pulse used in the imaging sequence disclosed in non-patent documents 1 and 2, then signals from fluid (magnetic resonance frequency of 128 MHz) in regions without a static magnetic field non-uniformity cannot be sufficient reduced. If the size of the excitation profile at +2.5 kHz is set as 1, then the excitation profile at the reference zero frequency will be about 1/1,000 of that size. The total signal from fluid at the reference zero frequency will be nearly 10,000 times larger than the total signal caused by protons near the cell labeled with magnetic nanoparticles.

Therefore in off-resonance imaging, the size of the excitation profile at the zero reference frequency is preferably less than 1/10,000 in the case that the size of the excitation profile at +2.5 kHz is 1. If not, then the desired signal will be buried within the signal from fluid at the zero reference frequency and detecting it will be impossible. Therefore in the imaging sequence disclosed in the non-patent documents 1 and 2, the size of the excitation profile at the zero reference frequency is set to less than 1/10,000 of the excitation profile size at +2.5 kHz, by combining the 90° pulse with the 180° pulse as a pair. The amplitude-modulated burst RF pulse designed under conditions shown in this embodiment has an excitation profile that been sufficiently reduced in size in the frequency region from a symmetrical waveform at the zero reference frequency (128 MHz). If the size of the excitation profile at +2.5 kHz is set as 1, then the excitation profile at the reference zero frequency will be less than 1/1,000 of that size. The size of the excitation profile at the zero reference frequency can be sufficiently reduced at just the 90° pulse, so there is no need to combine the 90° pulse and the 180° pulse into a pair. A slice can therefore be selected and a 180° pulse can be used the same as in conventional imaging.

A phase encoded magnetic field gradient 34 and a readout magnetic field gradient 33 are next applied and the magnetic resonance signal 36 is measured. The center frequency during signal measurement is set to the magnetic resonance frequency (128 MHz) of the hydrogen photon in fluid at 3 T. The pulse sequence is repeated for a specified number of times while changing the intensity of the phase encoded magnetic field gradient 34, and the data needed for reconstructing the image is acquired.

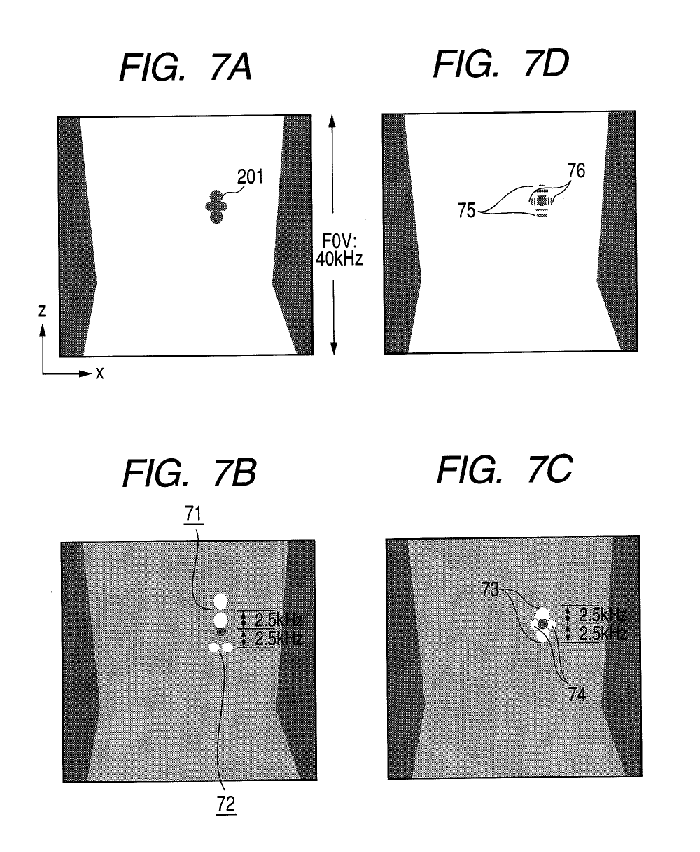

FIG. 7 is drawing showing these reconstruction images. FIG. 7A is a drawing of the state in FIG. 6A, or in other words, a drawing of the image captured by On-resonance imaging. The luminosity on the outer side and the inner side of the cell labeled with magnetic nanoparticles has declined and the image appears dark. FIG. 7B is a drawing of the image for the image captured in the state in FIG. 6B. The drawing is of off-resonance imaging so that the luminosity has declined in almost all sections of the subject and appears dark. The inner side of the cell labeled with magnetic nanoparticles contains large static magnetic non-uniformities so the luminosity is low and the image appears dark. The resonant frequency of protons along the same axis as the static magnet field on the outer side of cells labeled with magnetic nanoparticles is around +2.5 kHz and matches the carrier frequency of the amplitude-modulated burst RF pulse and is excited so that those sections appear as a high-luminosity area 71 on the screen. However, the resonant frequency of the protons near the outer periphery of the labeled cells is separated about +2.5 kHz from the center frequency during signal measurement so that the high-luminosity area 71 appears at a position shifted horizontally just +2.5 kHz as a frequency along the read-out direction. Converting this position into distance yields, (field-of-view [cm]×(2.5 [kHz]/measurement band [kHz])).

The resonant frequency of protons near the outer periphery of cells labeled with magnetic nanoparticles is near −2.5 kHz, and this section appears as high-luminosity area 72 on the screen after excitation. However, the protons near the outer periphery of the labeled cells is separated by about +2.5 kHz from the center frequency during signal measurement so that a high-luminosity area 72 appears at a position shifted horizontally just −2.5 kHz as a frequency along the read-out direction. Converting this position into distance yields, (field-of-view [cm]×(−2.5 [kHz]/measurement band [kHz])). FIG. 7C shows a high luminosity display of a region 73 shifted −2.5 kHz horizontally with high-luminosity area 71 set as a frequency along the read-out direction.

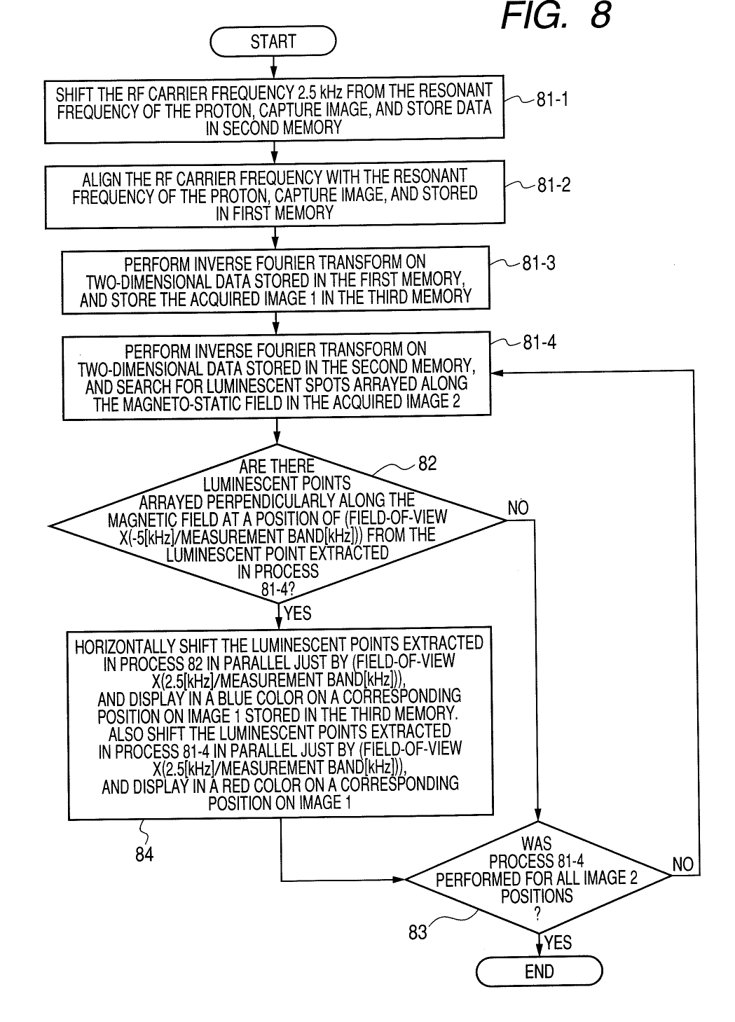

FIG. 7C also shows a high luminosity display of a region 74 shifted +2.5 kHz horizontally with high-luminosity area 72 as a frequency along the read-out direction. FIG. 7D shows in red, an area 75 at a position corresponding to the high luminosity region 73 in FIG. 7C, and in the On-resonance image of FIG.7A, and shows an image displayed in blue of an area 76 at a position corresponding to the high luminosity region 74 in FIG. 7C. FIG. 8 is a flow chart for showing the processing flow for detecting positions of cells labeled with magnetic nanoparticles. In the process in FIG. 8, a search is made for two bright points arrayed in a direction matching the direction of the static magnetic field on the screen, and a decision is made on whether (or not) there are two bright points arrayed intersecting a direction corresponding to the static magnetic field direction, within a 5 mm diameter of a position shifted −5 kHz horizontally as a frequency in a direction matching the read-out direction.

This value of −5 kHz is calculated as −2×((magnetic resonance frequency of protons at 3 T)×(20 ppm)). This 20 ppm is the characteristic value of the SPIO, and changes when the type of magnetic nanoparticle is changed. In other words, a decision usually made on whether there are two bright points arrayed intersecting the static magnetic field direction within a 5 mm diameter of a position shifted horizontally, from the two bright points arrayed in the direction of the static magnetic field by −2×(magnetic resonance frequency of protons at the MRI apparatus magnetic field strength)×(characteristic value of magnetic nanoparticles)). If there are two bright points arrayed intersecting the static magnetic field direction, then the bright points are identified as caused by cells labeled with magnetic nanoparticles. If two bright points are not arrayed intersecting the static magnetic field then these are identified as bright points are caused by a static magnetic non-uniformity unrelated to cells labeled with magnetic nanoparticles. This method is capable of detecting position of cells labeled with magnetic nanoparticles from one type of off-resonance image. The method of this invention is therefore capable of detecting positions of cells labeled with magnetic nanoparticles at a speed higher than the conventional method that requires two types of Off-resonance images.

An example for setting the carrier frequency (RF center frequency) of the amplitude-modulated burst RF pulse 31, from the magnetic resonance frequency (128 MHz) of protons in fluid at 3 T, to a frequency shifted plus 2.5 kHz was described above. However, the same method may be used to detect the positions of cells labeled with magnetic nanoparticles by setting the carrier frequency to a frequency shifted minus 2.5 kHz. In the process, a search is first made for two bright points arrayed intersecting the direction of the static magnetic field and then a decision is made whether there are two bright points arrayed intersecting the static magnetic field direction at a position shifted horizontally by 5 kHz as a frequency along the read-out direction. If there are two bright points then the bright points are identified as bright points caused by cells labeled with magnetic nanoparticles. If two bright points were not found, then the points are identified as caused by a static magnetic non-uniformity unrelated to cells labeled with magnetic nanoparticles.

Also, the center frequency during signal measurement may be set to a frequency shifted plus 2.5 kHz from the magnetic resonance frequency (128 MHz) of protons in fluid at 3 T and the measurement made. In this case, the two bright points arrayed in the direction of the static magnetic field and caused by cells labeled with magnetic nanoparticles, will appear on the screen at positions where the points are actually present. Two bright points arrayed intersecting the static magnetic field direction appear at a position shifted by −5 kHz in a frequency along the read-out direction. A search is first of all made for two bright points arrayed in the direction of the static magnetic field, and a decision is made whether there are two bright points arrayed intersecting the static magnetic field at a position shifted from the searched points horizontally by −5 kHz at a frequency along the read-out direction. If two bright points are present then those bright points are judged as caused by cells labeled with magnetic nanoparticles. If two bright points are not present then the bright point is judge as due to a static magnetic field non-uniformity unrelated to cells labeled with magnetic nanoparticles. If decided that the bright points are caused by cells labeled with magnetic nanoparticles, then a color display is shown on the on-resonance image that corresponds to the high-luminosity area of the off-resonance image.

In that case, the two bright points arrayed along the static magnetic field direction are displayed at that same position in a color display on the On-resonance image. However two bright points arrayed intersecting the static magnetic field direction must be set shown as a color display at a position shifted horizontally by +5 kHz as a frequency along the read-out direction. So in this case of bright points arrayed along the static magnetic field direction, the calculation for shifting the points horizontally along the read-out direction on the screen can be omitted to effectively shorten the processing time.

Also, making a search by image processing for two bright points arrayed intersecting the static magnetic field direction is not always necessary, when deciding whether there are two bright points in a direction intersecting the static magnetic field from there along the read-out direction and shifting the position horizontally by −5 kHz. In other words, the luminosity of the pixels in the area where the two bright points are supposed present on the image can be utilized to judge whether or not the bright points are caused by cells labeled with magnetic nanoparticles, by judging whether the luminosity is above a specified luminosity or below a specified luminosity. The luminosity thresholds can in this way be utilized not only to judge whether a bright point is caused by a cell labeled with magnetic nanoparticles but also to shorten the image processing time. In cases where bright point caused by cells labeled with magnetic nanoparticles, overlap unexpectedly here and there on bright points caused by static field non-uniformities unrelated to cells labeled with magnetic nanoparticles, attempting to clearly judge these points as two bright points may prove difficult. But in this case also, the threshold luminosity value of pixels in the area on the image where the two bright points are assumed present can be utilized to decide whether or not the bright points are caused by cells labeled with magnetic nanoparticles so that a stable identification can be made of high-luminosity signals caused by cells labeled with magnetic nanoparticles.

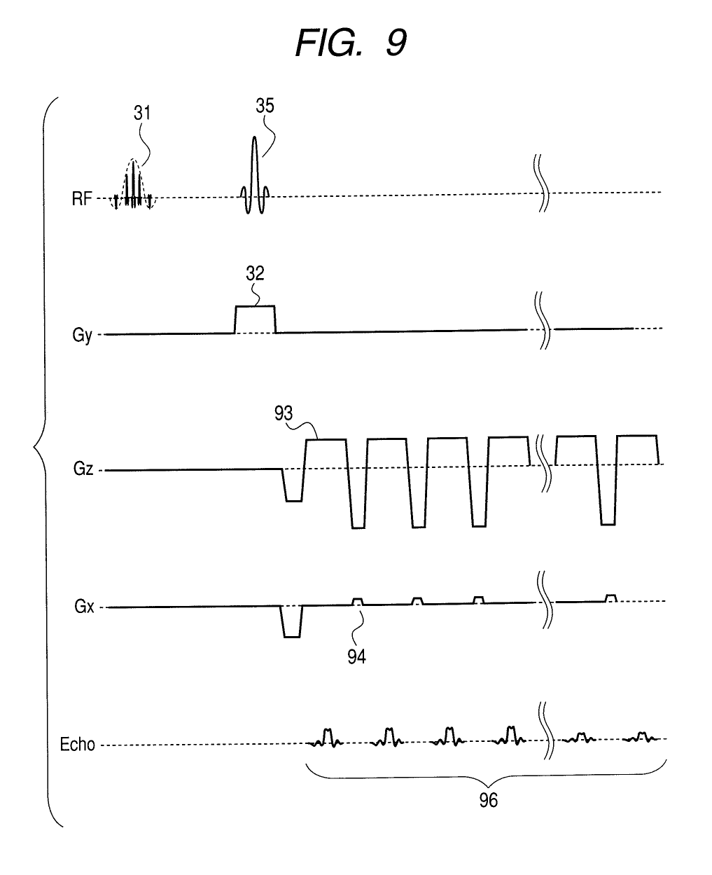

FIG. 9 is waveforms showing pulse sequence variations. The amplitude-modulated burst RF pulse 31 made up of five sub-pulses is first of all applied to the subject. The carrier frequency (RF center frequency) of the amplitude-modulated burst RF pulse 31, is set from the magnetic resonance frequency (128 MHz) of protons in fluid at 3 T, to a frequency shifted plus 2.5 kHz. After applying the amplitude-modulated burst RF pulse 31, a 180° pulse is applied along with a slice magnetic field gradient 32, the hydrogen proton within the desired cross section of the subject is inverted, and disperse the phase of the protons outside that cross section. Next, a phase-encoded magnetic field gradient 94 and an oscillating read-out magnetic field gradient 93 are applied, and the echo signal group (magnetic resonance signal group) 96 is measured. Data required for three-dimensional imaging can then also be acquired if a setting is made to insert a second phase encoding magnetic field gradient in the y direction. Utilizing this pulse sequence allows measuring multiple echo signals that were excited at one time, so that this pulse sequence offers the advantage that the time required for acquiring data needed for image reconstruction can be shortened compared to the pulse sequence shown in FIG. 3.

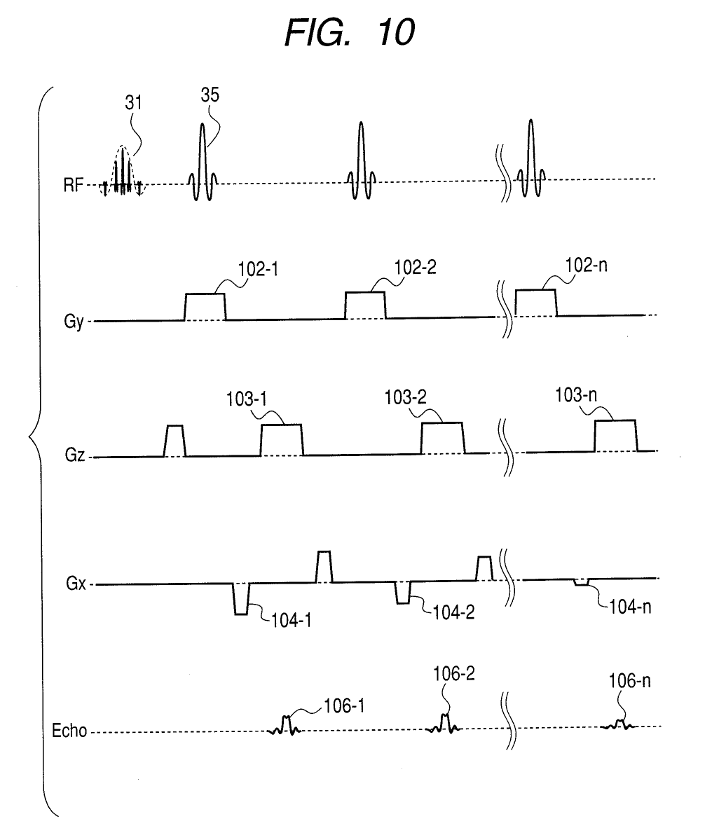

FIG. 10 is waveforms showing other pulse sequence variations. The carrier frequency (RF center frequency) of the amplitude-modulated burst RF pulse 31 is set to a frequency shifted plus 2.5 kHz from the magnetic resonance frequency (128 MHz) of protons in fluid at 3 T. After applying the amplitude-modulated burst RF pulse 31, a 180° pulse is applied along with a slice magnetic field gradient 102-1, the protons within the desired cross section of the subject are inverted, and disperse the phase of protons outside that cross section. Next, a phase-encoded magnetic field gradient 104-1 and a read-out magnetic field gradient 93 are applied, and the echo signal group (magnetic resonance signal group) 106-1 is measured. Further, a 180° pulse is applied along with a slice magnetic field gradient 102-2, the protons within the desired cross section of the subject are inverted, and then a read-out magnetic field gradient 103-2 applied along with a phase-encoded magnetic field gradient 104-2, and the echo signal 106-2 is measured. This step is repeated x times. Utilizing this pulse sequence allows measuring multiple echo signals that were excited at one time, so this pulse sequence offers the advantage that the time required for acquiring data needed for image reconstruction can be shortened compared to the pulse sequence shown in FIG. 3.

The imaging time is reduced so the cell position can be detected even in the case where a specified cell such as a labeled stem cell is moving comparative rapidly along with the movement of the bodily fluid. Moreover, the motion of the labeled cell can be recorded by installing a counting unit to count the time after administering the magnetic nanoparticles. The motion of the labeled cell can also be shown on as a moving image display on a display 20 based on the recorded time and the image. These labeled cells are not limited to stem cell and may for example also be macrophages labeled with magnetic nanoparticles. Macrophages tend to collect in inflamed sections within the body and so abnormalities in the body (such as arterial plaque and inflamed sections from early stage cancer) can be detected in their early stages by tracking the movements of these macrophages.

### Second Embodiment

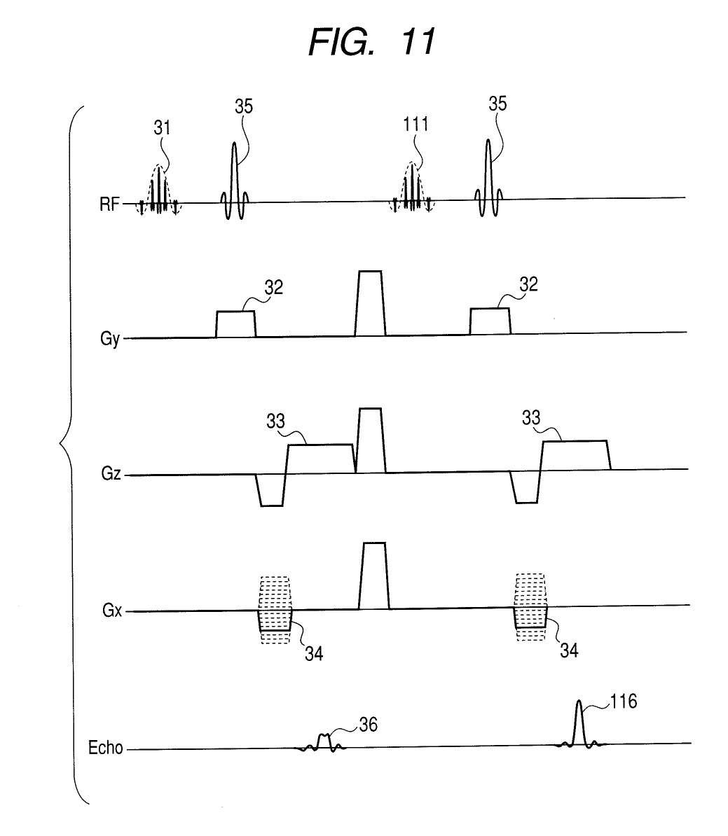

The pulse sequence used in this invention is described next while referring to FIG. 11. The horizontal axis in FIG. 11 is the time. An amplitude-modulated burst RF pulse 31 made up of five sub-pulses is first of all irradiated onto the subject. The carrier frequency (RF center frequency) of this amplitude-modulated burst RF pulse 31 is set to a frequency shifted plus 2.5 kHz from the magnetic resonance frequency (128 MHz) of the proton in the fluid at 3 T.

After applying an amplitude-modulated burst RF pulse 31, a 180° pulse is applied along with a slice magnetic field gradient 32, to invert the proton within the desired cross section of the subject and disperse the phase of the protons outside the cross section. A phase-encoded magnetic field gradient 34 and a read-out magnetic field gradient 33 are next applied and the echo signal group (magnetic resonance signal group) 36 is measured. The amplitude-modulated burst RF pulse 111 made up of five sub-pulses is first of all applied to the subject. The carrier frequency (RF center frequency) of the amplitude-modulated burst RF pulse 111 is set to a frequency shifted plus 2.5 kHz from the magnetic resonance frequency (128 MHz) of proton in fluid at 3 T. After applying the amplitude-modulated burst RF pulse 111, a 180° pulse is applied along with a slice magnetic field gradient 32, the proton within the desired cross section of the subject is inverted, and disperse the phase of the protons outside that cross section. A phase-encoded magnetic field gradient 34 and a read-out magnetic field gradient 33 are applied, and the echo signal group (magnetic resonance signal group) 116 is measured.

The pulse sequence is repeated for a specified number of times while changing the intensity of the phase encoded magnetic field gradient 34, and the data needed for reconstructing the image is acquired. The echo signal 36 acquired in the first half of the pulse sequence shown in FIG. 11, is data for reconstructing the Off-resonance image. The echo signal 116 acquired in the second half of the pulse sequence shown in FIG. 11, is data for reconstructing the On-resonance image. As described using FIG. 6, the frequency band excited by the amplitude-modulated RF pulse 31, and the frequency band excited by the amplitude-modulated RF pulse 111 do not mutually overlap on each other so that Off-resonance and On-resonance images can be consecutively imaged.

In conventional MRI imaging, after one excitation, a standby time of approximately 0.5 seconds is required to allow the magnetism to recover before excitation to capture the next cross section. If the pulse sequence as shown in FIG. 11 is used then data required for On-resonance imaging can be measured during the Off-resonance imaging standby time so that time is efficiently utilized. In other words, this pulse sequence possesses the advantage that positions of cells labeled with magnetic markers can be detected at high speed. A 180° pulse is also applied in the first half of the pulse sequence shown in FIG. 11 to render the effect that fatty signals are suppressed in the On-resonance image acquired in the latter half of the of the pulse sequence. Images where this fatty signal is suppressed are effective for diagnosis of tumors. This method allows acquiring both Off-resonance images and On-resonance images in one imaging, and can therefore detect positions of cells labeled with magnetic particle with both high-speed and high-accuracy. Positions of cells labeled with magnetic nanoparticles can be identified even when moving at comparatively high speed and consequently an MRI apparatus utilizing a stable functional contrast agent can be provided that contains a function for tracking specified cells or for early stage cancer diagnosis.

### Third Embodiment

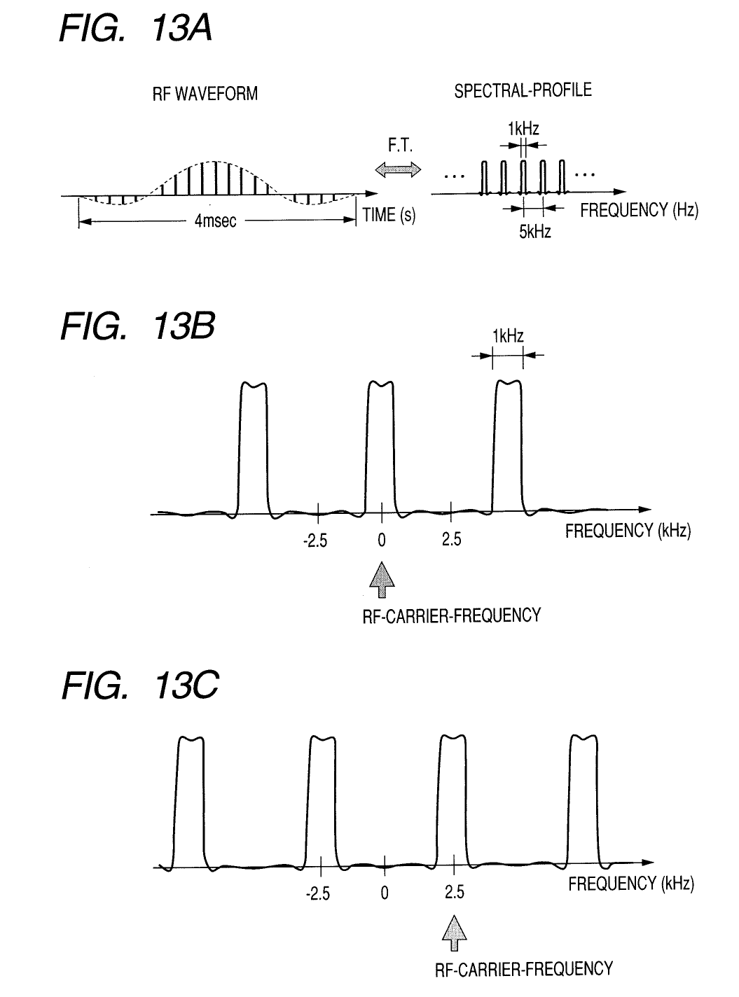

The amplitude-modulated burst RF pulse utilized in this embodiment is described next while referring to FIG. 13. The drawing on the left side in FIG. 13A shows a waveform of the time region while the figure on the right side shows a profile of the frequency region where the time region waveform was subjected to Fourier transform. In FIG. 13A, the interval in the comb type profile within the frequency range is to 5 kHz, and the frequency band for one profile of the comb profile is designed to be one kHz. FIG. 13B and 13C are enlarged drawings of the right figure (spectral profile) in FIG. 13A, and the horizontal axis is the frequency. Using a magnetic resonance frequency (128 MHz) for the proton in fluid as a reference of zero, the frequency shift from that reference zero is shown on the horizontal axis and the extent of excitation on each frequency is shown on the vertical axis. As shown in FIG. 13B, during imaging (On-resonance) imaging, when the center frequency (carrier frequency) is set to a magnetic resonance frequency (128 MHz) of proton in fluid at 3 T, the nucleus of the hydrogen atoms is excited within a range from −0.5 kHz to 0.5 kHz. The protons possessing a magnetic resonance frequency within a range from −4.5 kHz to −0.5 kHz and within a range from 0.5 kHz to 4.5 kHz are not excited.

In other words, those protons in areas near the outer side of cells labeled with magnetic nanoparticles are not excited. On the other hand, as shown in FIG. 13C, when the center frequency (carrier frequency) is set to a magnetic resonance frequency +2.5 kHz of proton in fluid at 3 T and imaging (Off-resonance imaging) performed, the protons possessing a magnetic resonance frequency in a range from −3 kHz to −2 kHz and +2 kHz to 3 kHz are excited. However, the protons possessing a magnetic resonance frequency within a range from −2 kHz to +2 kHz are not excited. In other words, the protons in areas near the outer side of cells labeled with magnetic nanoparticles are not excited. Compared to the excitation state shown in FIG. 6, the frequency range excited in FIG. 13B and 13C has become sharper at each 2.5 period.

Magnetic non-uniformities occur inside an actual body even at boundaries with air pockets, and deviations in the magnetic resonance frequency of the proton can occur from even just these non-uniformities. In off-resonance imaging, signals with high luminosity can also be detected at positions where the excitation RF frequency accidentally matches the magnetic resonance frequency. When the frequency range of the excitation becomes sharp as shown in FIG. 13, there are fewer occurrences of the RF excitation frequency accidentally matching the magnetic resonance frequency of protons in regions where magnetic non-uniformities occur such as at boundaries with air. Since occurrences of high luminosity signals caused by cells labeled with magnetic nanoparticles can be reduced in Off-resonance imaging, this embodiment renders the effect that processing time required for identifying high luminosity signals caused by cells labeled with magnetic nanoparticles from among other high luminosity signals can be reduced.

### Fourth Embodiment

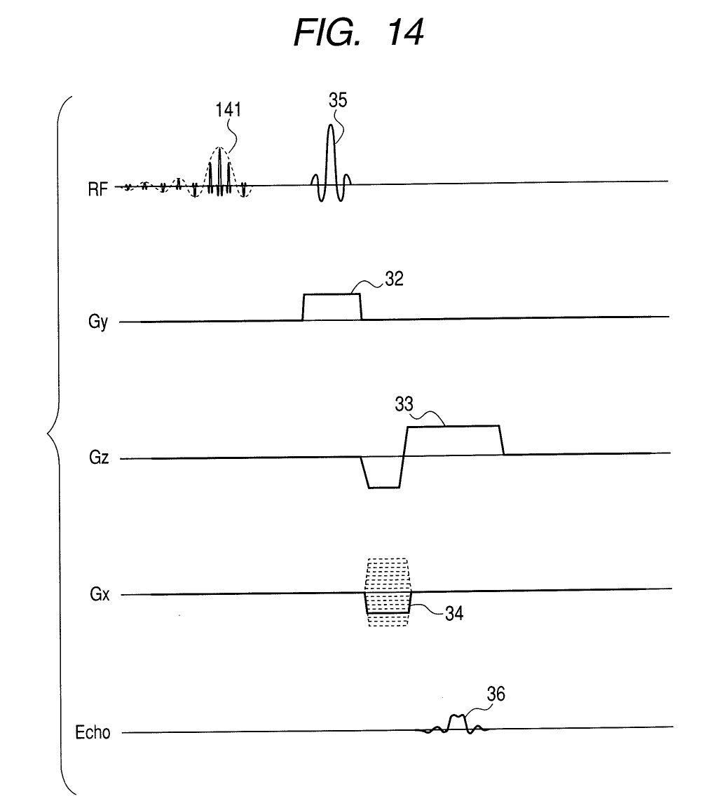

The pulse sequence used in this invention is described next while referring to FIG. 14. The horizontal axis in FIG. 14 is the time. A bilaterally symmetrical amplitude-modulated burst RF pulse 141 made up of nine sub-pulses is first of all irradiated onto the subject. The carrier frequency (RF center frequency) of this amplitude-modulated burst RF pulse 141 is set to a frequency shifted plus 2.5 kHz from the magnetic resonance frequency (128 MHz) of the proton in the fluid at 3 T. After applying the amplitude-modulated burst RF pulse 31, a 180° pulse is applied along with a slice magnetic field gradient 32 to invert the proton within the desired cross section of the subject and disperse the phase of the protons outside the cross section.

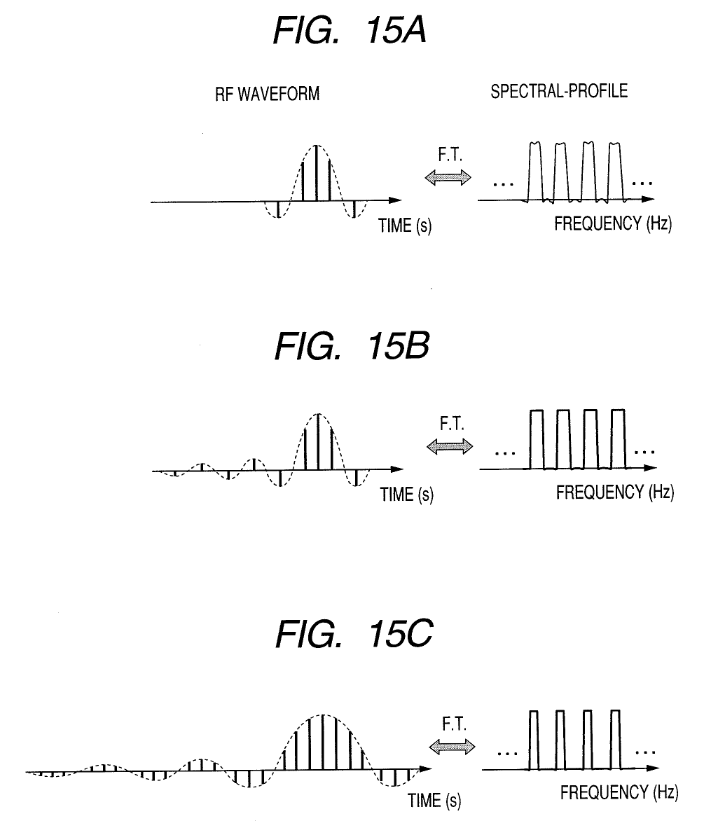

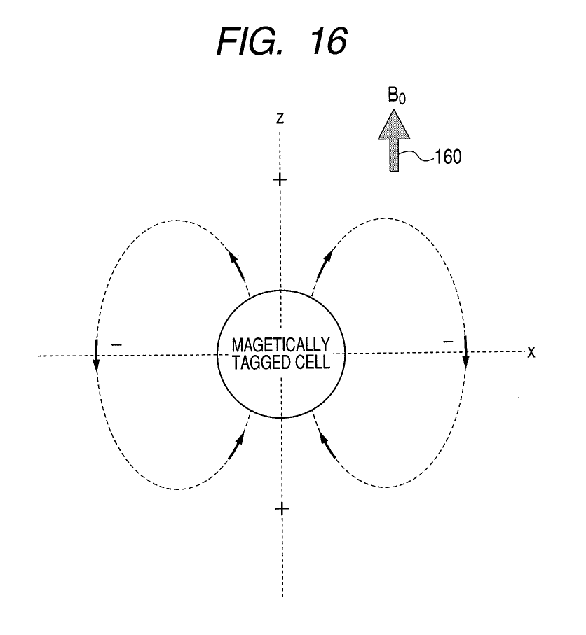

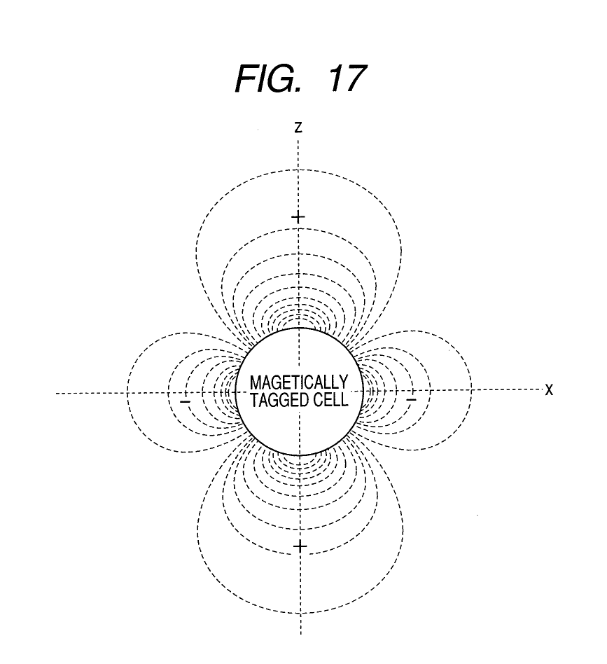

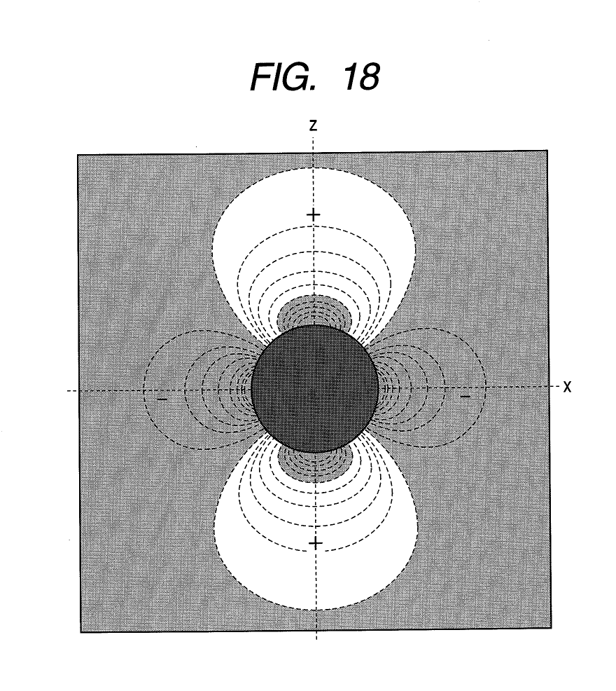

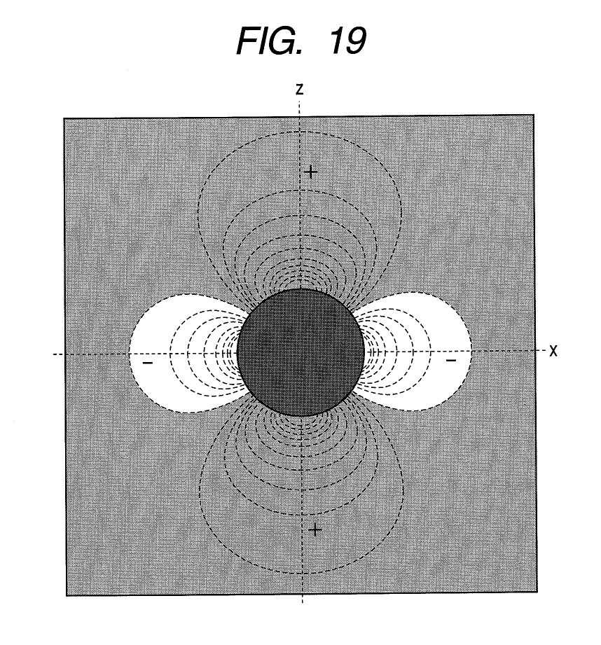

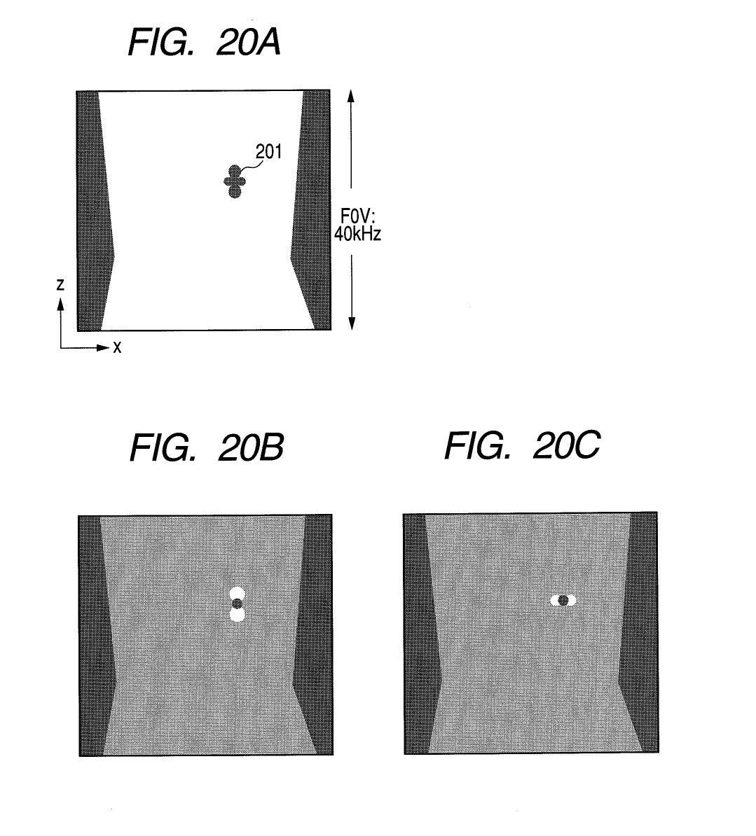

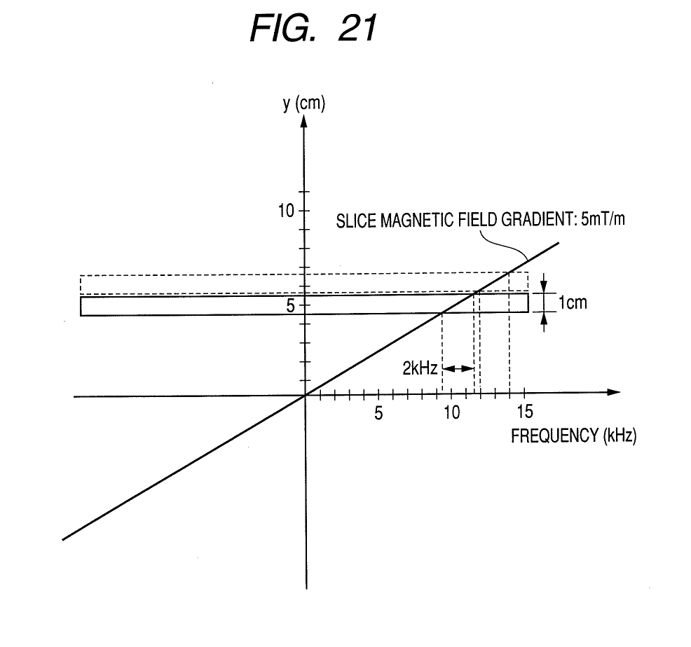

A phase-encoded magnetic field gradient 34 and a read-out magnetic field gradient 33 are next applied and the echo signal 36 is measured. The advantages rendered by utilizing the bilaterally symmetrical amplitude-modulated burst RF pulse shown in FIG. 14 are described while referring to FIG. 15. FIG. 15A shows a profile of bilaterally symmetrical amplitude-modulated burst RF pulse containing five sub-pulses as in the first embodiment, taken along the frequency axis after Fourier conversion.

FIG. 15B shows a profile of the bilaterally symmetrical amplitude-modulated burst RF pulse of this embodiment made up of nine sub pulses, when subjected to Fourier conversion along the frequency axis. Comparing the profiles on the frequency axes in FIG. 15A and FIG. 15B reveals that the peaks and dips in FIG. 15B are closer to a rectangular shape. This rectangular shape allows higher accuracy in identifying high luminosity signals caused by labeled cells because the rectangular shape lowers the intensity distortion of the image. The more the wave number of the sinc function for the amplitude-modulated burst RF pulse increases on the time axis, the more the profile on the frequency axis approaches a rectangular shape. As the wave number of the sinc function increases, the profile will of course approach a rectangular shape on the frequency axis for the bilaterally symmetrical amplitude-modulated burst RF pulse, however the main lobe (center of the sinc function) amplitude-modulated burst that is symmetrical on left and right, and the 180° pulse time period will become longer. The longer main lobe and pulse period cause the problem that they make it impossible to measure the signal for the short echo time.

To resolve this problem, the wave number for the sinc function on the side opposite the pulse can be increased and made bilaterally symmetrical, to make the profile along the frequency axis approach a rectangular shape, and moreover allows measuring the signal for the short echo time. FIG. 15C shows an example where the period of the sinc function of the envelope for the bilaterally symmetrical amplitude-modulated burst RF pulse envelope was lengthened. In this case, the frequency excitation range becomes narrower. Magnetic non-uniformities occur inside an actual body even at boundaries with air pockets, and deviations in the magnetic resonance frequency of the proton can occur from even just these non-uniformities. In off-resonance images, signals with high luminosity might be detected at positions where the excitation RF frequency accidentally matches the magnetic resonance frequency. Narrowing the frequency excitation range narrows, lowers the occurrence of the excitation RF frequency accidentally matching the magnetic resonance frequency of protons in regions where magnetic non-uniformities occur such as at boundaries with air. This embodiment therefore renders the effect that the processing time required for identifying luminance signals caused by cells labeled with magnetic nanoparticles from other high luminosity signals can be shortened.

The features that are characteristic of this invention were described above. However other variations may be employed including: setting of a first frequency based on information about magnetic nanoparticles loaded from the magnetic particle information storage unit, and the magnetic resonance frequency of the proton at the magnetic field strength of the static magnetic field of the MRI apparatus, and also setting the carrier frequency of the amplitude-modulated burst RF pulse to a second frequency shifted substantially (magnetic resonance frequency of the proton at the magnetic field strength of the static magnetic field of the MRI apparatus) from the first frequency, and, by detecting two bright points arrayed along the direction of the static magnetic field on the image, and detecting two bright points arrayed in a direction intersecting the direction of the static magnetic field on the image, and at a position from the first two bright points shifted horizontally −2×(the first frequency) on a frequency in the readout direction on images captured using an amplitude-modulated burst RF pulse as the excitation pulse, to detect the position of cells labeled with magnetic nanoparticles with high-speed and high-accuracy. The cell position can be identified even if the labeled cell is moving at comparatively high speed. Consequently the effect is rendered that an MRI apparatus utilizing a stable functional contrast agent can be provided that contains a function for tracking specified cells or for early stage cancer diagnosis. The magnetic nanoparticles are not limited to SPIO and any contrast agent utilizing a T2 short-term effect such as magnetite (Fe304) may be utilized. The intensity of the static magnetic field is not limited to 3 T (tesla) and may be a magnetic field strength used in MRI apparatus such as 1.5 T.

Besides utilization in MRI apparatus, the apparatus and imaging method of this invention may also be applied to analytic equipment using electromagnetic waves at frequencies from several megahertz to several gigahertz.

What is claimed is:

1. A magnetic resonance imaging apparatus comprising:
a magnet for generating a static magnetic field;
a transmitter coil for generating excitation RF pulses to apply to the subject placed in the static magnetic field;
a magnetic field gradient generator unit for generating a magnetic field gradient overlapping onto the static magnetic field;
a receiver coil for detecting nuclear magnetic resonance signals emitted from the subject;
a magnetic nanoparticle information storage unit for storing information about the magnetic nanoparticles;
a control unit for controlling the transmitter coil to generate amplitude-modulated burst RF pulses as excitation RF pulses by forming multiple high-frequency magnetic field sub-pulses separated time-wise whose amplitude is modulated by a function that repeatedly inverts the polarity and changes the amplitude at each polarity inversion, and for setting the time interval of the amplitude-modulated burst RF pulses to effectively 1/(2×a first frequency), and for setting the carrier frequency of the amplitude-modulated burst RF pulses to a second frequency substantially shifted from the resonant frequency of the proton at the magnetic field strength of the static magnetic field, by an amount equal to the first frequency; and
an information processor unit for forming image data based on the nuclear magnetic resonance signal detected by the receiver coil,
wherein the control unit sets the first frequency based on information about magnetic nanoparticles loaded from the magnetic nanoparticle information storage unit, and the magnetic resonance frequency of the proton at the magnetic field strength of the static magnetic field.

2. The magnetic resonance imaging apparatus according to claim 1, wherein the control unit sets the first frequency by multiplying the magnetic resonance frequency of the proton of the static magnetic field by the magnetic nanoparticle information loaded from the magnetic particle information storage unit.

3. The magnetic resonance imaging apparatus according to claim 1, wherein, the information processor unit detects two bright points arrayed along the direction of the static magnetic field on the image, and two bright points arrayed in a direction intersecting the direction of the static magnetic field on the image, and at a position from the first two bright points shifted horizontally −2×(the first frequency) of the frequency along the read out direction.

4. The nuclear magnetic resonance measurement apparatus according to claim 1, wherein the function is an asymmetrical Sinc function.

5. The nuclear magnetic resonance measurement apparatus according to claim 1, wherein the control unit controls a transmitter coil so as to set the carrier frequency of the amplitude-modulated burst RF pulse to the magnetic resonance frequency of the proton at the magnetic field strength of the static magnetic field, after controlling the transmitter coil to set the carrier frequency of the amplitude-modulated burst RF pulse to the second frequency.

6. The nuclear magnetic resonance measurement apparatus according to claim 1, wherein immediately after measuring the magnetic resonance signal utilized for reconstructing the off-resonance image, the receiver coil measures the magnetic resonance signal utilized for reconstructing the on-resonance image acquired after setting the carrier frequency of the amplitude-modulated burst RF pulse to the magnetic resonance frequency of the proton at the magnetic field strength of the static magnetic field.

7. The nuclear magnetic resonance measurement apparatus according to claim 1, wherein the magnetic nanoparticles are iron oxide.

8. The nuclear magnetic resonance measurement apparatus according to claim 1, wherein the magnetic nanoparticles are magnetite.
